(19) United States
(12) Patent Application Publication
(10) Pub. No.: US 2009/0180999 A1
(43) Pub. Date: Jul. 16, 2009

(54) METHOD OF PREVENTING, CONTROLLING AND AMELIORATING URINARY TRACT INFECTIONS USING CRANBERRY DERIVATIVE AND D-MANNOSE COMPOSITION

(75) Inventors: John A. Minatelli, Sanford, FL (US); W. Stephen Hill, Ocala, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: U.S. Nutraceuticals, LLC d/b/a Valensa International, Eustis, FL (US)

(21) Appl. No.: 12/348,947

(22) Filed: Jan. 6, 2009

Related U.S. Application Data
(60) Provisional application No. 61/020,558, filed on Jan. 11, 2008.
(60) Provisional application No. 61/023,905, filed on Jan. 28, 2008.

(51) Int. Cl. A61K 35/74; A61K 36/45; A61K 31/352

(52) U.S. Cl. 424/93.45; 424/732; 514/456

(57) ABSTRACT

A method is disclosed that prevents, controls and ameliorates urinary tract infections caused by E-coli by administering a therapeutically effective amount of a human dietary supplement composition comprising a cranberry derivative or proanthocyandin containing concentrate and D-mannose combined in a form suitable for oral consumption. The cranberry derivative or proanthocyandin containing concentrate comprises from about one percent (1.0%) by weight to about 95 percent (95.0%) by weight on a dry weight basis. The composition is formulated for delivering, a D-mannose unit dosage between about 0.5 to about 5.0 grams per dose and further comprising a diuretic and wherein the composition formed from the cranberry derivative or proanthocyandin containing concentrate, D-mannose and diuretic are effective together for binding to E-coli to aid in flushing the E-coli from the urinary tract system while preventing binding of E-coli to cells in the urinary tract system.

### RELATED APPLICATIONS

This application is based upon prior filed provisional application Ser. No. 61/020,558 filed Jan. 11, 2008 and provisional application Ser. No. 61/023,905 filed Jan. 28, 2008.

### FIELD OF THE INVENTION

The present invention relates preventing, controlling and ameliorating urinary tract infections (UTI) using cranberry derivative and D-Mannose compositions.

### BACKGROUND OF THE INVENTION

Urinary tract infections (UTIs) are generally defined as the presence (>100,000/mL) of bacteria in the urine. UTIs are commonly caused by Gram-negative bacteria, particularly Escherichia coli (E-Coli), and infect primarily women. This infection is enabled by the adherence and colonization of bacteria to urinary tract epithelial cells. Adherence by E. coli is performed by proteinaceous fibers (fimbriae) on the bacteria cell wall, which attach to specific oligosaccharide receptors on uroepithelial cells. Antibiotics are commonly prescribed for treatment, but may promote bacterial resistance. One in four women will also have a recurrence of the infection. Natural substances which could treat and prevent UTIs could be useful for those suffering this condition.

Consumption of cranberries has been found to be effective in preventing UTIs. Cranberry products can prevent adhesion of bacteria to uroepithelial cells in the urinary tract, thereby reducing the ability of the bacteria to create an infection (DiMartino et al., “Reduction of Escherichia Coli Adherence to Uroepithelial Bladder Cells After Consumption of Cranberry Juice: A Double-Blind Randomized Placebo-Controlled Cross-Over Trial,” World Journal of Urology 2006); (Liu et al., “Role of Cranberry Juice on Molecular-Scale Surface Chraacteristics and Adhesion Behavior of Escherichia Coli,” Biotechnology Bioenineering, 2006). Proanthocyanidins (condensed tannins) found in the cranberry juice have been shown to inhibit E. coli adherence (Howell et al., “Inhibition of the Adherence of P-Fimbricated Escherichia Coil to Uroepithelial-Cell Surfaces by Proonthecyandin Extracts from Cranberries,” Journal of Medicine, 1998). Some E. coli fimbriae bind specifically to mannose (a sugar). D-mannose is not metabolized by humans, but if consumed, will enter the bladder and cause the bacterial fimbriae to attach to the D-mannose, rather than the urinary tract cells. This allows the body to flush bacteria from the body. To mitigate existing UTIs and prevent recurrence, regular consumption of cranberry and D-mannose will prevent bacteria from adherence, colonization and infection. For this strategy to work, consumer compliance is necessary. D-mannose has a natural sweetness, and cranberry juice and its derivatives possess an acceptable flavor.

Combinations as compositions using cranberry have been presented by others, for example, in GB2396811 (WO200405638), the disclosure which is hereby incorporated by reference in its entirety. As noted in that reference, that composition includes an extract from a plant that is a member of the Ericaceae, Rosaceae, Pinaceae or Vitaceae family and at least one sugar that is not metabolized or is only partly metabolized by the human or animal body. The sugar is preferably a monosaccharide such as L-arabinose, L-fucose, D-mannose, L-rhamnose, L-xylose, lyxose or galactose. A preferred composition includes an extract of cranberry with D-mannose. These compositions may be used to treat bacterial infection caused by El coli, particularly urinary tract infections (UTI's). Compositions also include an anthocyanidin or a proanthocyanidin and at least one sugar that is not metabolized or is only partly metabolized by the human or animal body are also described.

Example of D-mannose compositions are also disclosed in U.S. Patent Publication Nos. 2007/0166292 and 2007/0244069; and U.S. Pat. Nos. 5,525,341 and 6,210,681, the disclosures which are hereby incorporated by references in their entirely.

### SUMMARY OF THE INVENTION

Cranberry derivatives and D-mannose are combined in a novel and unobvious concentration and proportion with various additives for preventing, controlling and ameliorating urinary tract infections caused by E-coli by administering a therapeutically effective amount of a human dietary supplement composition as a cranberry derivative or proanthocyandin containing concentrate and D-mannose combined in a form suitable for oral consumption. The cranberry derivative or proanthocyandin containing concentrate is formed from about one percent (1.0%) by weight to about 95 percent (95%) by weight on a dry weight basis and formulated for delivering a D-mannose unit dosage between about 0.5 to about 5.0 grams per dose. At least one diuretic additive is added such that the composition is effective for binding to the E-coli to aid in flushing the E-coli

In one aspect, the D-mannose is derived from a natural or synthetic source. The cranberry derivative can be derived from whole cranberries, juice, puree, extract, dried powder concentrate, seed extract, or any combination thereof. A carrier can be administered for example, maltodextrin, starch, cellulose, food-grade silicas or flow agents, on one or more acidulants for exampole citric acid, malic acid or ascorbic acid.

In another aspect, the composition is prepared and packaged in a wet or dry form suitable for direct addition to water. Other food ingredient components can be added to increase the palatability of the formula, including, for example, natural and/or artificial flavors, nutritive and/or non-nutritive sweeteners, salts, acids or other suitable food ingredients. The composition can be incorporated into a solid or semi-solid food or a beverage. In one aspect, the composition is added to a liquid as a ready-to-drink beverage. The composition in another aspect can be provided as tablets, capsules, powders, emulsions, liquid concentrates, beverages, confectionary candies or liquid gels.

Other biologically active extracts and compounds can be added, including for example, vitamins, minerals, antioxidants, dietary fibers, tocopherols, tocotrienols, phytosterols, polysaccharides, polyphenolics or bioflavonoids. The composition can contain a naturally occurring diuretic including, for example, saw palmetto, juniper berry, pipsissewa leaf, horsetail herb, cornsilk, uva ursi, asparagus root, goldenrod flowering tips, marshmallow leaf, parsley, black elderberry, peppermint, cleavers, buchu, dandelion, gravelroot, hydrangea, kava, linden, mullien, violet, or burdock or any combination thereof. The composition can contain a prescription diuretic, for example, chlorothiazide, furosemide, theobromine, theophylline, oleandrin, or amiloride.

A capsule can contain the cranberry derivatives and D-mannose and other additives in an effective dosage form. The composition can be formed into a roller compacted powder to increase bulk density and decrease effective dose volume.

The proanthocyandin containing concentrate can be derived from blueberry, grape, French maritime bark extract or D-mannose or any combination thereof. In another aspect a probiotic can be added, for example lactobacillus spp. that competes and has activity against undesirable bacteria, including E coli. Examples of lactobacillus spp. include acidophilus, rhamnosus, reuteri, casei or sporogenes spp. In another aspect, other additives could include Oregon grape (mahonia aquifolium), honey bee pollen (apis Mellifica), myrrh (commiphora spp.), hops (humulus lupus), plantain leaf (plantago spp.), or marshmallow root (althaea offcinalis). Golden seal (hydrastis Canadensis) can be added for increasing IgM production and has been found effective for use in the treatment method and the composition. Stinging nettle (Urtica dioica) can be added as a diuretic and has been found effective for use in the treatment method and the composition. Echinacea (echinecea spp.) can be added for stimulating anti-inflammatory effects and immunomodulatory and immunostimulant activity and has been found effective for use in the treatment method and composition.

### DETAILED DESCRIPTION OF THE PREFERRED EMBODIMENTS

The present invention will now be described more fully hereinafter, in which preferred embodiments of are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

D-Mannose is a simple sugar and related as a (steroisomer) to glucose. It is metabolized by the human body and in small amounts helps maintain a healthy urinary tract. This occurs because of the interaction between D-Mannose and the bacteria found in many bladder infections known as Escherichia Coli “E.Coli”. The urinary tract infection occurs when E.Coli sticks to the inner walls of the bladder and often reaches as far as the kidneys. The cell walls of the E.Coli are covered with tiny finger like projections as in amino acid-sugar complex forming a “glycoprotein”, also referred to as a “lectin”. The D-Mannose sticks to the E.Coli lectins better than it sticks to human cells unless the large quantity of D-Mannose when taken internally spills into the urine through the kidneys and coats any E.coli so that they no longer stick to the inside walls of the bladder and urinary tract. The E.Coli's are rinsed with minimal urination.

Some believe this is better than an antibiotic that will kill not only the unwanted micro-organisms but also kill friendly micro-organisms. For example many females suffer from yeast infections following antibiotic use because the friendly bacteria are killed along with the bad bacteria, leaving antibiotic insensitive yeast to grow and reproduce. Long-term antibiotic use can lead to major disruptions in normal body microflora and disrupts health.

It has been found that the D-Mannose can be helpful in these situations. In accordance with a non-limiting aspect of the present invention, the use of a cranberry derivative or proanthocyandin containing concentrate in combination with D-Mannose and a diuretic and other effective additives in a therapeutically effective amount with proper proportions is a suitable composition for oral consumption and strengthens the effect of D-Mannose. The method as described with the effective composition including the described combination and additives provides for improved vaginal health and bridge the gap between the anus and urethra. For example, the cranberry derivative or proanthocyandin containing concentrate can be about one percent by weight to about 95 percent by weight on a dry weight basis and the total composition formulated for delivering a D-mannose unit dosage between 0.5 and 5.0 grams per dose. Although cranberry does contain some D-Mannose the supplemental composition containing the cranberry derivative or proanthocyandin containing concentrate or multiple cranberry derivative(s) and D-Mannose with a diuretic is effective and advantageous as a drink when each are proportioned in a specific manner.

In one non-limiting aspect, the cranberry derivative(s) are derived from whole cranberries, juice, puree, extract, dried powder concentrate, seed extract, or any combination thereof.

The composition is incorporated with a suitable carrier such as maltodextrin, starch, cellulose, food-grade silicas, flow agents, and one or more acidulants such as citric acid, malic acid and ascorbic acid in a non-limiting example. The composition as a formula can be prepared and packaged in a wet or dry form suitable for direct addition to water and form a beverage drink. Other additives to the drink can be used.

In one non-limiting and preferred aspect, the composition as a formulation delivers a D-mannose unit dosage between about 0.5 and about 5.0 grams per dose. As another non-limiting example, the cranberry derivative or proanthocyandin containing concentrate typically comprises from about one percent (1.0%) by weight to about 95 percent (95.0%) by weight of the formula on a dry weight basis. This proportion is therapeutically effective and advantageous.

Other components can be added to increase the palatability of the formula, including for example, natural and/or artificial flavors, nutritive and/or non-nutritive sweeteners, salts, acids or other suitable food ingredients. The compositions can be incorporated into food and beverage, for example, as a human dietary supplement composition in a ready to drink beverage.

The compositions can be in the form of tablets, capsules, powders, emulsions, liquid concentrates, beverages, confectionary candies and liquid gels. Other biologically active extracts and compounds can be added including for example, vitamins, minerals, antioxidants, dietary fibers, tocopherols, tocotrienols, phytosterols, polysaccharides, polyphenolics and bioflavonoids and have been found to add to the therapeutically effectiveness of the treatment method and composition.

The formula as a composition can contain a naturally occurring diuretic such as saw palmetto, juniper berry, pipsissewa leaf, horsetail herb, cornsilk, uva ursi, asparagus root, goldenrod flowering tips, marshmallow leaf, parsley, black elderberry, peppermint, cleavers, buchu, dandelion, gravelroot, hydrangea, kava, linden, mullien, violet, burdock and the like and extracts of such and in different combinations.

The formula as a composition can also contain a prescription diuretic such as chlorothiazide, furosemide, theobromine, theophylline, oleandrin, amiloride and the like.

Additionally, the formulation may be compacted in suitable roller compaction device in order to increase the bulk density, thereby reducing the consumption volume needed for an effective dose. It is known that consumer dose compliance decreases with increasing capsule size and number, therefore formulation compaction can increase consumer compliance and resulting effectiveness, particularly in capsule presentations. For example, the composition can have a higher density, resulting in an effective therapeutic dosage using two capsules instead of four capsules when capsules are used in the method of treatment.

The proanthocyandin containing concentrate can be derived from blueberry, grape, French maritime bark extract or D-mannose or any combination thereof. In another aspect a probiotic can be added, for example, lactobacillus spp. that competes and has activity against undesirable bacteria, including E coli. Examples of lactobacillus spp. include acidophilus, rhamnosus, reuteri, casei or sporogenes spp. Oregon grape (mahonia aquifolium), honey bee pollen (apis Mellifica), myrrh (commiphora spp.), hops (humulus lupus), plantain leaf (plantago spp.) , or marshmallow root (althaea offcinalis). Golden seal (hydrastis Canadensis) can be added for increasing IgM production. Stinging nettle (Urtica dioica) can be added as a diuretic. Echinacea (echinecea spp.) can be added for stimulating anti-inflammatory effects and immunomodulatory and immunostimulant activity.

Probiotics as added and described above are operative as beneficial live microorganisms and assist the body's naturally occurring gut flora to reestablish themselves and aid in managing lactose intolerance, lowering cholesterol and blood pressure, improving immune function and preventing infections, improving effective with helicobacter pylori and antibiotic-associated diarrhea, reducing inflammation, improving mineral absorption, irritable bowel syndrome and colitis and preventing harmful bacterial growth under stress. Common forms can be synbiotics, including use with prebiotics and formed for example from dairy products. They have antibiotic effects and reduce inflammation.

Probiotics when used with the compostion can prevent and treat inflammatory bowel disease. Probiotic clones with direct immunomodulatory activity possibly could have anti-inflammatory effects in the intestine. Murine-derived probiotic lactobacilli have been shown to inhibit TNF-α secretion. Probiotic effects could result from direct modulation of mucosal inflammatory responses.

Increasing Immunoglobulin M (IgM) production is beneficial as an antibody and can form polymers where multiple immunoglobulins are covalently linked together with disulfide bonds, for example, a pentamer or hexamer. It diffuses well and is typically found in the interstitium in low quantities and effective at complement activation.

As noted above, goldenseal acts to increase IgM and similar components include Mahonia (Oregon grape) and Berberis (Barberry). It is believed that these components have the ability to inhibit drug resistance efflux pumps (MDR pumps) of bacteria. Goldenseal contains isoquinoline alkaloids: hydrastine, berberin, berberastine, hydrastinine, tetrahydroberberastine, canadine, and canalidine. Possibly the action of 8-oxotetrahydrothalifenine is operative Berberine and hydrastine are believed to act as quaternary bases.

Proanthocyanidins as identified above can also be found in many plants, for example, apples, pine bark, cinnamon, grape seed, cocoa, grape skin, and red wines of vitis vinifera. Bilberry, cranberry, black currant, green tea, black tea and other plants also contain these flavonoids. The berries of chokeberry, such as black chokeberry, have high concentrations of proanthocyanidin and can be used.

Proanthocyanidins can act as vasoactive polyphenols such as in red wine and reduce the risk of coronary heart disease. They are believed to suppress production of a protein endothelin-1 that constricts blood vessels. Proanthocyanidins are believed to have antioxidant activity and stabalize collagen maintenance of elastin—two critical proteins in connective tissue that support organs, joints, blood vessels, and muscle. Proanthocyanidins are also believed to reduce histamine production and beneficial in treating allergies while also improving circulation by strengthening capillary walls. They are also believed to inhibit enzymes that break down collagen and help collagen repair and act as a sunscreen. Proanthocyanidins can cross the blood-brain barrier to fight free radicals in the vessels of the brain and increase mental acuity.

As to the diuretic action of stinging nettle, some studies show that a sex hormone binding globulin (SHEG), aromatase, epidermal growth factor and prostate steroid membrane receptors are involved in the anti-prostatic effect It is not clear that 5α-reductase or androgen receptors are used. Some anti-inflammatory activity may be effected by extract and a polysaccharide fraction. It is believed also to contain different acids, for example, silicic, threonic, and formic acids and contain soime amines such acetylcholine, choline, serotonine and histamine as well as flavonoids. A polysaccharide fraction could also aid in an anti-inflammatory effect and polypeptide fraction. Hops contain alpha- and beta-acids, where the alpha-acids occur as humulone, cohumuline and adhumulone, and essential oils and prenyulflavonoids.

Hops can have a sedative effect and also act as a potent estrogen receptor agonist in estrogen-responisive cells and aid in treating menstrual symptoms Bitter acids in hops have an antibacterial and antifungal activity, Marshmallow root is believed to have bactericidal and anti-inflammatory properties. Myrhh is typically found as an oleo-gum and includes a volatile oil of sesquiterpenes, sterols, and steroids and can be used for antiparasitic effects and infections for females and males. It is believed that honey bee pollen contains some Apalbumin1 (Apa1) as a royal jelly (RJ) and honey glycoprotein having various biological properties. It could possibly stimulate macrophages to release tumor necrosis factor alpha (TNFalpha) and possibly has immunostimulatory effects. Plantain leaf is useful in GI therapy and treats hperlipidemia through various actions. It includes various acids with various flavonoids.

Oregon Grape has various alkaloids, including berberine, berbamine, canadine, and hydrastine and can treat diarrhea in patients infected with E. coli. Such as by slowing the transit time in the intestine and possibly inhibiting the ability of bacteria to attach to human cells, which helps prevent infections in the intestines and urinary tract, Echinacea has antibacterial properties possibly selectively modulates the catalytic activity of CYP3A at hepatic and intestinal sites.

Many modifications and other embodiments of the invention will come to the mind of one skilled in the art having the benefit of the teachings presented in the foregoing description. Therefore, it is understood that the invention is not to be limited to the specific embodiments disclosed, and that modifications and embodiments are intended to be included within the scope of the appended claims.

1. A method of preventing, controlling and ameliorating urinary tract infections caused by E-coli by administering a therapeutically effective amount of a human dietary supplement composition comprising a cranberry derivative or proanthocyandin containing concentrate and D-mannose combined in a form suitable for oral consumption, wherein the cranberry derivative or proanthocyandin containing concentrate comprises from about one percent (1.0%) by weight to about 95 percent (95.0%) by weight on a dry weight basis and said composition is formulated for delivering a D-mannose unit dosage between about 0.5 to about 5.0 grams per dose and further comprising a diuretic and wherein the composition formed from the cranberry derivative or proanthocyandin containing concentrate, D-mannose, and diuretic are effective together for binding to E-coli to aid in flushing the E-coli from the urinary tract system while preventing binding of E-coli to cells in the urinary tract system.

2. The method of claim 1, in which the D-mannose is derived from a natural or synthetic source.

3. The method of claim 1, in which the cranberry derivative is derived from whole cranberries, juice, puree, extract, dried powder concentrate, seed extract, or any combination thereof.

4. The method of claim 1, and further comprising administering a carrier comprising maltodextrin, starch, cellulose, food-grade silicas or flow agents, on one or more acidulants comprising citric acid, malic acid or ascorbic acid.

5. The method of claim 1, and further comprising preparing and packaging the composition in a wet or dry form suitable for direct addition to water.

6. The method of claim 1, adding food components to increase palatability comprising natural or artificial flavors, nutritive or non-nutritive sweeteners, salts, or acids or any combination thereof

7. The method of claim 1, and further comprising incorporating the composition in a food or beverage.

8. The method of claim 7, and further comprising incorporating the composition into a ready to drink beverage.

9. The method of claim 1, and further comprises forming tablets, capsules, powders, emulsions, liquid concentrates, beverages, confectionary candies or liquid gels to which the composition is contained.

10. The method of claim 1, and further comprising adding biologically active extracts and compounds, comprising vitamins, minerals, antioxidants, dietary fibers, tocopherols, tocotrienols, phytosterols, polysaccharides, polyphenolics or bioflavonoids or any combination thereof.

11. The method of claim 1, wherein the diuretic comprises a naturally occurring diuretic.

12. The method according to claim 11, wherein said naturally occurring diuretic comprises saw palmetto, juniper berry, pipsissewa leaf, horsetail herb, cornsilk, uva ursi, asparagus root, goldenrod flowering tips, marshmallow leaf, parsley, black elderberry, peppermint, cleavers, buchu, dandelion, gravelroot, hydrangea, kava, linden, mullien, violet or burdock or any combination thereof.

13. The method of claim 1, wherein the diuretic comprises a prescription diuretic.

14. The method according to claim 13, wherein the prescription diuretic comprises chlorothiazide, furosemide, theobromine, theophylline, oleandrin or amiloride.

15. The method of claim 1, and further comprising adding an antibiotic.

16. The method of claim 1, and further comprising forming a capsule containing the cranberry derivative or proanthocyandin concentrate and D-mannose in an effective dosage form.

17. The method of claim 1, and further comprising roller compacting the cranberry derivative or proanthocyandin containing concentrate and D-mannose and a binder additive to increase bulk density and decrease effective dose volume.

18. The method according to claim 1, wherein the proanthocyandin containing concentrate is derived from blueberry, grape, French maritime bark extract or D-mannose or any combination thereof.

19. The method according to claim 1, and further comprising adding a probiotic as lactobacillus spp. that competes and has activity against undesirable bacteria, including E coli.

20. The method according to claim 19, wherein the step of adding lactobacillus spp. comprises adding acidophilus, rhamnosus, reuteri, casei or sporogenes spp.

21. The method according to claim 1, and further comprising adding Oregon grape (mahonia aquifolium), honey bee pollen (apis Mellifica), myrrh (commiphora spp.), hops (humulus lupus), plantain leaf (plantago spp.), or marshmallow root (althaea offcinalis).

22. The method according to claim 1, and further comprising adding golden seal (hydrastis Canadensis) for increasing IgM production.

23. The method according to claim 1, and further comprising adding stinging nettle (Urtica dioica) as a diuretic.

24. The method according to claim 1, and further comprising adding Echinacea (echinecea spp.) for stimulating anti-inflammatory effects and immunoinodulatory and immunostimulant activity.
